(19) United States
(12) Patent Application Publication
(10) Pub. No.: US 2017/0354594 A1
(43) Pub. Date: Dec. 14, 2017

(54) PROTEASE COMPOSITIONS FOR USE IN MODIFYING SEMEN

(71) Applicants: Marc L. Berger, Brookline, MA (US); David S. Newburg, Newtonville, MA (US)

(72) Inventors: Marc L. Berger, Brookline, MA (US); David S. Newburg, Newtonville, MA (US)

(21) Appl. No.: 15/526,310

(22) PCT Filed: Nov. 19, 2015
(86) PCT No.: PCT/US15/61518

§ 371 Date: May 11, 2017

Related U.S. Application Data
(60) Provisional application No. 62/081,813, filed on Nov. 19, 2014.

(51) Int. Cl. A61K 9/00; A23L 29/00; A61K 38/47; A61K 38/48; A23G 3/36

(52) U.S. Cl. CPC A61K 9/0056; A61K 38/4826; C12Y 304/22033; A61K 38/4886; A23G 3/366; A61K 38/4873; A61K 38/47; C12Y 304/21004; C12Y 304/24003; A23L 29/06; A23V 2002/00

(57) ABSTRACT

Provided herein are edible compositions for reducing semen viscosity and/or enhancing semen flavor, and methods of using and preparing such compositions.

### RELATED APPLICATION

This application claims the benefit under 35 U.S.C. §119(e) of U.S. provisional application number 62/081,813, filed Nov. 19, 2014, which is incorporated by reference herein in its entirety.

### BACKGROUND OF THE INVENTION

Negative perceptions and experiences are widely reported when semen is tasted and/or swallowed during sexual activity. The taste of semen is frequently described as bitter, harsh, unpleasant, and bleach-like. The viscous nature of semen may be described as mucus-like and also contributes to the negative perceptions of semen palatability.

### SUMMARY OF THE INVENTION

One aspect of the present disclosure is an edible composition for reducing semen viscosity. The composition comprises (a) one or more enzymes, which can be a protease and/or a glycosidase; and (b) a matrix that comprises a sweetening agent. The one or more enzymes can be embedded in or encapsulated by the matrix, and the matrix is dissolvable inside an oral cavity, thereby releasing the one or more enzymes.

In any of the edible compositions described herein, the one or more enzymes may be one or more proteases. Exemplary proteases include, but are not limited to, collagenase, bromelain, trypsin, and chymotrypsin. Alternatively, the one or more enzymes may be one or more glycosidases. Examples of glycosidase include, but are not limited to, amylase, sucrase, maltase, lactase, fucosidase, galactosidase, N-acetylglucosidase, N-acetylgalactosidase, N-acetylneuraminidase, N-glycolylneuraminidase, sialidase, and mannosidase. In some examples, the one or more enzymes in the edible composition can be a combination of different proteases, a combination of different glycosidases, or a combination of protease and glycosidase.

In some embodiments, the amount of the one or more enzymes in the edible composition ranges from about 0.1 mg to about 300 mg. For example, the amount of the one or more enzymes in the edible composition may range from about 2 mg to about 50 mg, or from about 4 mg to about 40 mg.

In any of the edible compositions described herein, the sweetening agent can be sucrose, fructose, glucose, galactose, maltose, sorbitol, xylitol, lactitol, maltitol, erythritol, isomalt, a sugar substitute, or a combination thereof. In some embodiments, the sugar substitute can be stevia, aspartame, sucralose, neotame, acesulfame potassium, saccharin, or a combination thereof. The amount of the sweetening agent in the edible composition may range from about 50 mg to about 2 g.

In some embodiments, the matrix in the edible composition may consist of the sweetening agent. In other embodiments, the matrix may comprise at least one polymer, which is dissolvable in an oral cavity. Exemplary polymers include, but are not limited to, gelatin, pectin, cellulose, natural or synthetic gum, starch, or a combination thereof. In some examples, the matrix is a food product, e.g., candy, gummie, gel, fruit strip, or food bar.

In some examples, the sweetening agent is embedded in or encapsulated by the matrix, and dissolution of the matrix releases the sweetening agent. In some examples, the matrix is a film, which encapsulates the one or more enzymes. In some embodiments, both the one or more enzymes and the sweetening agent are embedded in the matrix.

Any of the edible compositions described herein may further comprise one or more flavoring agents. In some embodiments, the flavoring agent is embedded in or encapsulated by the matrix. The flavoring agent can be a natural flavoring agent, e.g., vanilla, chocolate, caramel, coffee, honey, molasses, milk, cream, butter, seeds, nuts, mint, fruit, herbs, vegetables, meats, citrus, and berries. Alternatively, the flavoring agent can be an artificial flavoring agent, e.g., isoamyl acetate, benzaldehyde, ethyl butyrate, allyl hexanoate, methyl anthranilate, ethyl propionate, limonene, ethyl maltol, ethylvanillin, ethyl decadienoate, methyl salicylate, chocolate, vanilla, caramel, and coffee. The amount of the flavoring agent in the edible composition may range from about 0.001 mg to about 200 mg.

In another aspect, the present disclosure provides a method of reducing seminal viscosity. The method comprises contacting any of the edible compositions described herein with a semen sample under conditions allowing for dissolution of the matrix to release the one or more enzymes. In some embodiments, the contacting step is performed by placing the edible composition in an oral cavity. In some embodiments, the oral cavity is a human oral cavity.

In some embodiments, the amount of the one or more enzymes in the composition is effective in reducing semen viscosity by at least 20%.

In yet another aspect, the present disclosure provides a method for preparing an edible composition as described herein. The methods comprise providing one or more enzymes described herein and encapsulating or embedding the one or more enzymes with a film, which is dissolvable in an oral cavity. In some embodiments, the method further comprises mixing a sweetening agent with the one or more enzymes prior to the encapsulating or embedding step.

Any of the films described herein may be used in the preparation method. In some embodiments, the film may comprise one or more of the sweetening agents as described herein, and optionally at least one polymer, which is dissolvable in an oral cavity, e.g., those described herein. In some embodiments, the film consists of the one or more sweetening agent.

### DETAILED DESCRIPTION OF THE INVENTION

In addition to spermatozoa, seminal fluid or semen is comprised of ions (potassium, magnesium, sodium, zinc, calcium, etc.), citrate, fructose, glucose, and protein. Though there is substantial variation in the concentration of each of the components among individuals, semen is generally considered protein-rich. A number of studies have found that the total protein concentration of semen is between 3700 mg/mL to 7460 mg/mL (Owen and Katz, J. Androl. 2005: 26(4): 459-469). Following ejaculation the seminal fluid quickly coagulates into a thick, gel-like material due to the activity of vesiculase released by the prostate (Walker, G. Bulletin of the Johns Hopkins Hospital, 1910, 21:182). The coagulated semen eventually liquefies over the course of several minutes (Owen and Katz, J. Androl. 2005: 26(4): 459-469). The semen coagulation process has been hypothesized to be a method for semen retention in the female reproductive tract, though its physiological role is not fully understood (Cohen, Adv. In Assisted Repro. Technol. 1990: 443-452).

During oral sexual activity, the viscosity of the ejaculated semen is reported to provide a negative perception of the experience. The taste of semen also contributes to this perception and is frequently described as bitter, harsh, unpleasant, and bleach-like. Efforts to improve the palatability of semen during oral sexual activity have been focused on changes to the individual's diet and various products, none of which substantially negate the viscosity or enhance the taste of the semen.

The present disclosure provides compositions and methods for reducing viscosity of semen and improving its taste.

I. Compositions

The edible compositions provided herein are designed to reduce the viscosity of semen and improve its taste when in contact with semen in, e.g., an oral cavity. The composition may comprise one or more enzymes and a matrix, which comprises one or more sweetening agents, and optionally one or more flavoring agents. The matrix is dissolvable inside an oral cavity to release the one or more enzymes and sweetening agents.

The compositions are configured such that the one or more enzymes and the sweetening agent are either embedded in or encapsulated by the matrix, and in some embodiments, the one or more sweetening agents constitute the matrix. As used herein, the term “edible composition” refers to a composition that can be consumed by an individual (e.g., a human) orally and is intended to be consumed by the individual. An edible composition may be ingested and/or chewed by an individual.

As used herein, the term “embed” or “embedded” refers to a substance (e.g., an enzyme and/or a sweetening agent) that is fixed or implanted in another substance (e.g., a matrix). In some embodiments, a substance may be fully embedded in another substance. In other embodiments, a substance may be partially embedded in another substance.

As also used herein, the term “encapsulate” or “encapsulated” refers to a substance (e.g., an enzyme and/or a sweetening agent) that is surrounded, enclosed, or coated by another substance (e.g., a matrix). In some embodiments, a substance may be fully encapsulated by another substance. In other embodiments, a substance may be partially encapsulated by another substance.

The matrix of the edible compositions provided herein is dissolvable inside an oral cavity (e.g., in a human oral cavity) to release the one or more enzymes and other components in the composition, such as the sweetening agent(s) and/or the flavoring agent(s). When in contact with semen in an oral cavity, the released one or more enzymes are able to perform their enzymatic function resulting in a reduction in the viscosity of the semen. The released sweetening agent(s) and flavoring agent(s) would improve the taste of the semen in the oral cavity.

The edible composition may be formulated in a suitable form for the intended use as described herein, including, but not limited to, a tablet, capsule, strip, film, gel, bar, gum, gummie, jelly, syrup, lozenge, pellet, paste, or wafer.

A. Enzyme

The edible compositions provided herein may include any enzyme that is capable of reducing the viscosity of semen or aiding in the reduction of semen viscosity, such as a protease, glycosidase, or combination thereof. Any enzyme that hydrolyzes or cleaves a chemical bond present in semen is compatible with the edible compositions described herein.

As used herein, the terms “protease,” “peptidase,” “proteinase,” and “proteolytic enzyme” can be used interchangeably throughout and refer to an enzyme that is capable of hydrolyzing a peptide bond between amino acids of a protein. Any protease that is capable of reducing the viscosity of semen can be used in the edible compositions described herein. A reduction in the viscosity of semen may also enhance the palatability of semen.

Proteases can be categorized based on the catalytic residue responsible for the protease function. For example, a protease may be a serine protease, threonine protease, cysteine protease, aspartate protease, glutamic acid protease, or metalloprotease. In some embodiments, the protease(s) contained in the edible composition can be a collagenase, bromelain, trypsin, chymotrypsin, or combination thereof.

In some embodiments, the protease is a collagenase. Collagenases are enzymes that are able to cleave the bond between a neutral amino acid and a glycine reside in motif Pro-X-Gly-Pro that is frequently found in the collagen protein. The collagenase may be a part of a crude collagenase mixture that comprises a collagenase as well as other proteolytic enzymes. In some embodiments, the collagenase is isolated from a mammal. Collagenases produced by mammals include collagenase-I, collagenase-II, and collagenase-III. The collagenase can be produced by a bacterium, such as Clostridium histolyticum. Bacterial collagenases can be categorized as class 1 or class 2 and may be able to cleave collagen protein at multiple sites. Class 1 collagenases typically initially cleave collagen near the periphery of the triple helix domain, whereas class 2 collagenase clave near the interior of the collagen protein.

In some embodiments, the protease is bromelain. Bromelain is a mixture of proteases that are extracted from plants belonging to the family Bromeliaceae. In one example, the bromelain is stem bromelain (EC 3.4.22.32). In another example, the bromelain is fruit bromelain (EC 3.4.22.33).

In some embodiments, the protease is a serine protease, such as trypsin (EC 3.4.21.4) or chymotrypsin (EC 3.4.21.1).

Alternatively or in addition, the one or more enzymes contained in the edible composition may be one or more glycosidases. As also used herein, the terms “glycosidase,” “glycosidic enzyme,” “glycoside hydrolase,” and “glycosyl hydrolase” can be used interchangeably and refer to an enzyme that is capable of hydrolyzing a glycosidic bond in an oligosaccharide or glycoconjugate such as a glycoprotein. Any glycosidase that is capable of reducing the viscosity of semen can be used in the edible compositions described herein. A reduction in the viscosity of semen may also enhance the palatability of semen.

Glycosidases can be classified based on the enzyme substrate specificity and/or mechanism of hydrolysis of the glycosidic bond. For example, α-glycosidases hydrolyze α-glycosidic linkages, and β-glycosidases hydrolyze β-glycosidic bonds. In other examples, N-linked glycosidases cleave N-linked glycosidic bonds, O-linked glycosidases cleave O-linked glycosidic bonds, and S-linked glycosidases cleave S-linked glycosidic bonds.

In some embodiments, the glycosidase is an amylase, sucrase, maltase, lactase, fucosidase, galactosidase, N-acetylglucosidase, N-acetylgalactosidase, mannosidase, neuramidase, hyaluraonidase, lysozyme, or a combination thereof. In some embodiments, the glycosidase is an amylase, such as an a-amylase (EC 3.2.1.1), β-amylase (EC 3.2.1.2), or a γ-amylase (EC 3.2.1.3). In some embodiments, the glycosidase is a sucrase such as a sucrase-isomaltase (EC 3.2.1.10), an invertase (EC 3.2.1.26), or a sucrose α-glycosidase (EC 3.2.1.48). In some embodiments, the glycosidase is maltase (EC 3.2.1.20). In some embodiments, the glycosidase is lactase (EC 3.2.1.23). In some embodiments, the glycosidase is a mannosidase such as a-mannosidase (EC 3.2.1.24) or β-mannosidase (EC 3.2.1.25). In some embodiments, the glycosidase is fucosidase (EC 3.2.1.51). In some embodiments, the glycosidase is galactosidase, such as β-galactosidase (EC 3.2.1.23). In some embodiments, the glycosidase is N-acetylglucosidase (EC 3.2.1.50), N-acetylgalactosidase, N-acetylneuramidase, N-glycolylneuraminidase, or any other sialidase.

In some embodiments, the edible composition described herein may comprise one enzyme, e.g., a protease or a glycosidase Alternatively, the edible composition may comprise more than one enzyme (e.g., 2, 3, 4, 5, or more enzymes). For example, the edible composition may comprise a combination of more than one (e.g., 2, 3, 4, 5, or more) proteases. In another example, the edible composition comprises a combination of more than one (e.g., 2, 3, 4, 5, or more) glycosidases. When desired, the edible composition may comprise a combination of at least one protease and at least one glycosidase.

Enzymes for use in the edible compositions described herein may be obtained from any source known in the art, for example, the enzymes may be purchased from a commercial source, synthesized, or isolated from a natural source. In one example, collagenase is isolated from Clostridium histolyticum. In another example, bromelain is isolated from a plant of the family Bromeliaceae. In yet another example, the amylase is prepared from yeast, fungi or any other suitable organism. In some examples, the enzymes may be prepared by recombinant technology.

The one or more enzymes may be present in the edible compositions described herein in any amount suitable to result in a reduction in the viscosity of semen. In some embodiments, the amount of the enzyme is 0.01-500 mg, 0.1-300 mg, 0.5-200 mg, 1-100 mg, 2-50 mg, 4-40 mg, 5-20 mg, or 10-15 mg. In some embodiments, the amount of the enzyme is about 0.01 mg, 0.05 mg, 0.1 mg, 0.5 mg, 1 mg, 5 mg, 10, mg, 15 mg, 20 mg, 25 mg, 30 mg, 40 mg, 50 mg, 60 mg, 70 mg, 80 mg, 90 mg, 100 mg, 125 mg, 150 mg, 175 mg, 200 mg, 225 mg, 250 mg, 275 mg, 300 mg, 350 mg, 400 mg, 450 mg, or 500 mg. In embodiments in which the edible compositions comprise more than one enzyme (e.g., 2, 3, 4, 5, or more enzymes), the total amount of the enzymes may be 0.01-500 mg, 0.1-300 mg, 0.5-200 mg, 1-100 mg, 2-50 mg, 4-40 mg, 5-20 mg, or 10-15 mg. In some embodiments, the total amount of the enzymes is about 0.01 mg, 0.05 mg, 0.1 mg, 0.5 mg, 1 mg, 5 mg, 10, mg, 15 mg, 20 mg, 25 mg, 30 mg, 40 mg, 50 mg, 60 mg, 70 mg, 80 mg, 90 mg, 100 mg, 125 mg, 150 mg, 175 mg, 200 mg, 225 mg, 250 mg, 275 mg, 300 mg, 350 mg, 400 mg, 450 mg, or 500 mg. In some embodiments, each of the enzymes is 0.01-500 mg, 0.1-300 mg, 0.5-200 mg, 1-100 mg, 2-50 mg, 4-40 mg, 5-20 mg, or 10-15 mg. In some embodiments, the amount of each of the enzymes is about 0.01 mg, 0.05 mg, 0.1 mg, 0.5 mg, 1 mg, 5 mg, 10, mg, 15 mg, 20 mg, 25 mg, 30 mg, 40 mg, 50 mg, 60 mg, 70 mg, 80 mg, 90 mg, 100 mg, 125 mg, 150 mg, 175 mg, 200 mg, 225 mg, 250 mg, 275 mg, 300 mg, 350 mg, 400 mg, 450 mg, or 500 mg.

In some embodiments, the amount of the enzyme(s) is the amount sufficient to reduce the viscosity of a semen sample by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or at least 50%.

In some embodiments, the one or more enzymes are embedded in the matrix. In embodiments in which the edible composition comprises more than one enzyme (e.g., 2, 3, 4, 5, or more enzymes), each of the enzymes may be embedded in the matrix. In some embodiments, one or more enzymes are embedded in the matrix and one or more enzymes are not embedded in the matrix. In some embodiments, the sweetening agent is embedded in the matrix. In some embodiments, the one or more enzymes and the sweetening agent are embedded in the matrix.

In some embodiments, the one or more enzymes are encapsulated by the matrix. In embodiments in which the edible composition comprises more than one enzyme (e.g., 2, 3, 4, 5, or more enzymes), each of the enzymes may be encapsulated by the matrix. In some embodiments, one or more enzymes are encapsulated by the matrix and one or more enzymes are not encapsulated by the matrix. In some embodiments, the sweetening agent is encapsulated by the matrix. In some embodiments, the one or more enzymes and the sweetening agent are encapsulated by the matrix.

B. Sweetening Agent

The edible compositions provided herein may include one or more suitable sweetening agents to increase the palatability of semen or enhance the taste of semen. As used herein, a “sweetening agent” refers to any agent that may be used to sweeten an edible composition. Suitable sweetening agents for use in making the edible compositions described herein may be naturally sweetening agents, which may be isolated from a natural source or synthesized via conventional methods. Alternatively, they may be an artificial or synthetic sweetening agent. In some embodiments, the sweetening agent is a sugar or sugar alcohol. In other embodiments, the sweetening agent is a sugar substitute, such as a high intensity sweetener.

Examples of sugars for use in the edible compositions described herein include, but are not limited to, sucrose, fructose, glucose, galactose, maltose, lactose, xylose, maltodextrin, corn syrup, or combinations thereof. In other examples, the sweetening agent can be a sugar substitute, such as a sugar alcohol or polyol.

Exemplary sugar alcohols include, but are not limited to, sorbitol, xylitol, glycerol, threitol, arabitol, ribitol, galactitol, fucitol, iditol, inositol, lactitol, maltitol, erythritol, isomalt, volemitol, maltotriitol, mannitol, maltotetraitol, polyglycitol, or a combination thereof.

Sugar substitutes for use in the edible compositions described herein may be natural sweetening agents (e.g., stevia). Alternatively, they may be artificial sweetening agents, including, but not limited to, aspartame, cyclamate, sucralose, neotame, acesulfame potassium, saccharin, advantame, or a combination thereof.

Additional sweetening agents compatible for use in the edible compositions described herein will be evident to one of skill in the art and can be found, for example, in U.S. Pat. No. 5,164,214, U.S. Pat. No. 4,921,939, U.S. Pat. No. 3,170,801, and PCT Publication No. WO 1992/007473 A1.

In some embodiments, the edible compositions described herein may comprise more than one sweetening agent (e.g., 2, 3, 4, 5, or more). The more than one sweetening agents may be of different types. For example, one sweetening agent may be a sugar and another sweetening agent may be a sugar substitute or an artificial sweetening agent.

Sweetening agents for use in the edible compositions described herein may be obtained from any source known in the art, for example, the sweetening may be purchased from a commercial source, synthesized, isolated, or extracted from a natural source.

The sweetening agent may be present in the edible compositions described herein in any amount suitable to enhance the taste of semen. In some embodiments, the amount of the sweetening agent is 5 mg-5 g, 15 mg-4 g, 30 mg-3 g, 50 mg-2 g, 100 mg-1 g, 250 mg-750 mg, or 350-500 mg. In some embodiments, the amount of the sweetening agent is about 25 mg, 50 mg, 75 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1 g, 1.5 g, 2 g, 2.5 g, 3 g, 3.5 g, 4 g, 4.5 g, or 5 g.

In some embodiments, the one or more enzymes are encapsulated by the matrix and the sweetening agent is embedded in the matrix. In some embodiments, the one or more enzymes are embedded in the matrix and the sweetening agent is encapsulated by the matrix.

C. Matrix

The edible compositions described herein contain a matrix, which is dissolvable in an oral cavity such as a human oral cavity to release the substances (e.g., the one or more enzymes and/or the one or more sweetening agents as described herein) embedded in or entrapped by the matrix. As used herein, a “matrix” refers to a material that may form a structure for the edible compositions or provide a delivery vehicle for the enzyme(s) and sweetening agent of the edible compositions. The matrix for use in the edible compositions described herein may be in a solid form. Alternatively, it may be in a semi-solid form or a viscous liquid. In some embodiments, the matrix is a film that encapsulates at least the one or more enzymes.

In some embodiments, the matrix is made of the one or more sweetening agents used in the edible compositions described herein. For example, the matrix may be made of a sugar, such as mannose, mannitol, dextrose, lactose, galactose, trehalose, corn syrup, or a cyclic glycan, such as cyclodextrin.

Alternatively, the matrix is made of a suitable material and encapsulates the one or more enzymes and the sweetening agent(s). Alternatively, the one or more enzymes and/or the sweetening agent(s) may be embedded in the matrix.

In some embodiments, the matrix may comprise a polymer that is dissolvable in an oral cavity. Examples include, but are not limited to, gelatin, pectin, cellulose, natural or synthetic gum, starch, modified starch, or a combination thereof. In some examples, the matrix is a gelatin, such as a non-mammalian gelatin. In other examples, the matrix is a natural gum, such as agar, alginic acid, sodium alginate, carrageenan, gum Arabic, gum ghatti, gum tragacanth, karaya gum, guar gum, locust bean gum, beta-glucan, chicle gum, dammar gum, glucomannan, mastic gum, spruce gum, tara gum, gellan gum or xanthan gum. Alternatively, the matrix can be a synthetic gum, or a pectin, such as a fruit pectin. In some examples, the matrix may be starch or modified starch.

In some embodiments, the matrix may comprise at least one component selected from fruit concentrate, corn syrup, dried corn syrup, sugar, partially hydrogenated cottonseed oil, citric acid, acetylated monoglycerides, fruit pectin, dextrose, malic acid, and vitamin C (ascorbic acid). In some embodiments, the at least one component selected from fruit concentrate, corn syrup, dried corn syrup, sugar, partially hydrogenated cottonseed oil, citric acid, acetylated monoglycerides, fruit pectin, dextrose, malic acid, and vitamin C (ascorbic acid) is embedded in or encapsulated by the matrix.

The matrix of any of the edible compositions described herein may be a food product, which may be formed in such a way to encapsulate or embed the enzyme and/or sweetening agent. Such a food product may be a candy, confectionary, gummie, gel, fruit strip, or food bar. A candy may include soft or semi-solid candies such as caramels, or hard candies. In some embodiments, the matrix is a fruit strip. Fruit-by-the-foot® is an example of a fruit strip that may be used in the edible compositions. Exemplary food bars include PowerBars® and Cliff Bars®.

In some embodiments, the matrix of the edible composition comprises more than one component, which may be of the same type or of different types. For example, the matrix may comprise a polymer such as fruit pectin and a sweetening agent such as sugar or corn syrup.

In some embodiments, the matrix material is configured such that it allows temporary or short-term affixation of the edible composition to the roof of the oral cavity or the back of the teeth.

Selection of the matrix material may also depend on the desired rate of dissolution inside an oral cavity. In some embodiments, the matrix is a quick dissolving matrix. In some embodiments, the matrix is a slow dissolving matrix.

D. Flavoring Agents

Any of the edible compositions described herein further comprise one or more flavoring agent. As used herein, a “flavoring agent” refers to a component that provides a flavor to an edible composition. The flavoring agent may be embedded in the matrix or encapsulated by the matrix of the edible composition. Any flavoring agent known in the art may be compatible with the edible compositions provided herein. In some embodiments, the flavoring agent further enhances the palatability or taste of semen.

The flavoring agent of the edible compositions may be any natural flavoring agent or artificial flavoring agent. A natural flavoring agents may be any flavoring agent that occurs in nature. Naturally flavoring agents may be obtained from a natural source or may be synthesized. Examples of natural flavoring agents include vanilla, chocolate, caramel, coffee, honey, molasses, milk, cream, butter, seeds, nuts, mint, fruit, herbs, vegetables, meats, citrus, and berries. An artificial flavoring agent may be any flavoring agent that does not occur in nature but may mimic the favor of a natural flavoring agent. Artificial flavoring agents may be synthesized using any method known in the art. Examples of artificial flavoring agents include isoamyl acetate, benzaldehyde, ethyl butyrate, allyl hexanoate, methyl anthranilate, ethyl propionate, limonene, ethyl maltol, ethylvanillin, ethyl decadienoate, methyl salicylate, caramel, and vanillin.

The edible compositions may comprise any combination of two or more flavoring agents, such as two or more natural flavoring agents, two or more artificial flavoring agents, or one or more natural flavoring agents and one or more artificial flavoring agents. In some embodiments, the flavoring agents are chocolate and caramel.

The one or more flavoring agent may be present in the edible compositions described herein in any amount suitable to enhance the taste of semen. In some embodiments, the amount of the flavoring agent(s) is 0.0001 mg-500 mg, 0.0005 mg-450 mg, 0.001 mg-400 mg, 0.005 mg-350 mg, 0.01 mg-300 mg, 0.05 mg-250 mg, 0.1 mg-200 mg, 0.5 mg-150 mg, 1 mg-100 mg, 5 mg-50 mg, 10 mg-25 mg, 100 mg-500 mg, 100 mg-200 mg, or 50 mg-150 mg. In some embodiments, the amount of the flavoring agent(s) is about 0.0001 mg, 0.0005 mg, 0.001 mg, 0.005 mg, 0.01 mg, 0.05 mg, 0.1 mg, 0.5 mg, 1 mg, 5 mg, 10 mg, 20 mg, 30 mg, 40 mg, 50 mg, 60 mg, 70 mg, 80 mg, 90 mg, 100 mg, 200 mg, 300 mg, 400 mg, or 500 mg.

Any of the edible compositions described herein may also contain one or more preservatives, coloring agents, fillers, supplements, vitamins, and/or minerals.

II. Methods of Use

The edible compositions described herein can be used in methods for reducing seminal viscosity, and/or enhancing semen taste. As used herein, a “semen sample” refers to an amount of semen released by ejaculation. In some embodiments, the semen sample is from a human male. To perform the method described herein, the edible compositions may be contacted with a semen sample under conditions allowing for dissolution of the matrix to release the enzyme(s). Dissolution of the matrix may also release the sweetening agent. The contacting step may be performed by placing the edible composition in an oral cavity, such as a human oral cavity. For example, the edible composition is placed in the oral cavity prior to the initiation of oral sex or during oral sex. The edible compositions may be contacted with the semen sample in the oral cavity. In some examples, the edible compositions are contacted with the semen sample in a human oral cavity. Alternatively, the edible composition is contacted with a semen sample ex vivo, for example in a test tube.

The conditions that allow for dissolution of the matrix may be the presence of saliva or salivary enzymes, and/or the temperature in the oral cavity. The conditions that allow for dissolution of the matrix may also include mechanical disruption of the matrix, such as by chewing activity. In some embodiments, the matrix is partially dissolved to an extent sufficient to allow the enzyme(s) to exert its enzymatic activity so as to reduce the viscosity of a semen sample. In some embodiments, the matrix is partially dissolved, to an extent sufficient to release the enzyme(s) and allow the enzyme(s) to reduce the viscosity of a semen sample. In other embodiments, the matrix is fully dissolved.

Any method known in the art can be used to measure or evaluate the viscosity of a semen sample. In some embodiments, the semen viscosity is measured using a viscometer. In other embodiments, the semen viscosity is evaluated by a human subject. It should be appreciated that the viscosity of a solution (e.g., a semen sample) can be evaluated by the sensory system in the oral cavity.

The compositions and methods provided herein result in reducing the viscosity of a semen sample. In some embodiments, the semen viscosity is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or at least 50% as compared to a semen sample that has not been contacted with the compositions described herein.

III. Methods of Preparation

Also provided herein are methods of preparing the edible compositions described herein. The methods involve providing one or more enzymes, such as one or more protease, glycosidase or combination thereof, and encapsulating or embedding the enzyme(s) in a matrix (e.g., a film) that is dissolvable in an oral cavity. In some embodiments, a sweetening agent is further encapsulated or embedded in the matrix. In some embodiments, the sweetening agent is the film that encapsulates or embeds the enzyme(s).

The methods may further involve forming the matrix or the film of the edible composition into a desired shape and/or size. In some embodiments, the prepared edible composition is formed into a desired shape and/or size. In some embodiments, the edible compositions are formed in an appropriate shape and/or size for affixing the composition in the oral cavity, such as to the roof of the oral cavity or behind the teeth. In some embodiments, the prepared edible composition has a length and width of about 3 cm by 3 cm, about 2 cm by 3 cm, about 2 cm by 2 cm, about 1 cm by 3 cm, about 1 cm by 2 cm. In some embodiments, the prepared edible composition has a thickness of less than about 5 mm, such as about 1 mm, 2 mm, 3 mm, 4 mm, or 5 mm.

Without further elaboration, it is believed that one of skill in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

The following experiments aimed to determine whether a combination of a protease and a sweetening agent is able to reduce the viscosity and improve the taste of semen. Collagenase and fructose were combined in the presence or absence of an additive, as shown in Table 1, and brought to a volume of 1 mL with filtered water. 100 μL of semen was added to each of the combinations or to 1 mL of filtered water without collagenase or fructose as a control. Following incubation for 1 minute, the combinations were orally sampled and evaluated for viscosity, qualitative taste, and sweetness by 2 independent human volunteers. The averages of the evaluations between the volunteers are presented in Table 1.

TABLE 1

| Collagenase | Fructose | Additive | Viscosity | | |
|---|---|---|---|---|---|
| mg/mL | mg/mL | mg/mL | 1 = highest | Qualitative taste | Sweetness |
| 0 | 0 | 0 | 6 | Sticky, bitter | 0 |
| 1 | 0 | 0 | 8 | Less sticky, less bitter | 0 |
| 1 | 5 | 0 | 8 | Less sticky, less bitter | 0 |
| 1 | 5 | 3.3 Agar | 6 | Little sweet, bitter aftertaste | 2-3 |
| 1 | 5 | 3.4 xanthan gum | 4 | Sperm taste, bitter | 0 |
| 1 | 5 | 5.9 cetyl alcohol | 6 | bitter | 0 |

Addition of 1 mg/mL collagenase successfully reduced the viscosity of the semen samples in the presence or absence of fructose, as compared to the control sample. Collagenase also did not introduce any overt flavors. The addition of additives such as agar, xanthan gum, or cetyl alcohol counteracted the viscosity-lowering capacity of collagenase. Each of the semen samples containing a combination that included an additive was assessed as equally viscous or more viscous than the control semen sample.

The dose response relationship of collagenase and fructose was then evaluated to determine the amount of the protease and sweetening agent necessary to reduce the viscosity and enhance the taste of the semen samples.

Collagenase and fructose were combined as shown in Table 2 and brought to a volume of 1 mL with filtered water. 100 μL of semen was added to each of the combinations or to 1 mL of filtered water without collagenase or fructose as a control. Following incubation for 1 minute, the combinations were orally sampled and evaluated for viscosity, qualitative taste, and sweetness by independent human volunteers (Table 2).

TABLE 2

| Collagenase | Fructose | Viscosity | | |
|---|---|---|---|---|
| mg/mL | mg/mL | 1 = highest | Qualitative taste | sweetness |
| 0 | 0 | 1, 3 | Bitter, back | 0 |
| | | | of tongue | |
| 1 | 0 | 5, 6 | Bitter, sharp | 0 |
| | | | on tongue | |
| 1 | 5 | 5, 7 | No bitter, | 2, 5 |
| | | | sweet | |
| 3 | 15 | 6, 8.5 | A little sweet | 4, 8 |
| | | | and pleasant | |
| 10 | 50 | 9, 8 | Very sweet, | 6, 9 |
| | | | pleasant | |

Increasing the amount of collagenase in the combination reduced the viscosity of the semen samples. Addition of 10 mg/mL collagenase eliminated the sensation of viscosity. Similarly, increasing the amount of fructose in the combination enhanced the taste of the semen samples with the addition of 50 mg/mL fructose resulting in a taste described as “very sweet, pleasant.”

The amount of each of collagenase and fructose were further evaluated using the combinations shown in Table 3.

TABLE 3

| Collagenase | Fructose | Viscosity | | |
|---|---|---|---|---|
| mg/mL | mg/mL | 1 = highest | Qualitative taste | Sweetness |
| 0 | 0 | 6 | Bitter | 0 |
| 5 | 25 | 8 | Sweet | 8 |
| 10 | 50 | 9 | Moderately sweet | 9 |
| 15 | 75 | 9.2 | Sugary sweet, nice | 9.5 |
| 10 (22 hr rt) | 50 | 8.8 | Moderately sweet | 8.5 |

### Example 2

Oral delivery matrices for the protease and sweetening agent were evaluated for their duration of presence in the oral cavity and release and function of the protease.

### Artificially Flavored Fruit Strips

Artificially flavored and sweetened fruit strips were cut into a square shape of 2 cm length by 2 cm width by 2 mm. 8 mg of collagenase powder was added to the center of the strip, and the strip was folded in half with all borders pinched to provide a dry, dark environment for the collagenase. The 1 by 2 cm device made of a fruit strip surrounding collagenase was refrigerated until 20 min prior to use.

Following initiation of oral sex, the device was placed behind the front teeth of a human subject and secured by running the tongue against the device at the initiation of oral sex. Upon ejaculation, the fruit strip matrix was at least partially dissolved by running the tongue over the device, releasing the collagenase and eliminating the viscosity of the semen. The taste of the device was described as sweet, clean and fruity.

The efficacy of compositions prepared using various enzymes are shown in Table 4.

TABLE 4

| Enzyme | Viscosity | | |
|---|---|---|---|
| (8 mg/mL) | 1 = highest | Qualitative taste | Sweetness |
| Amylase (F) | 10 | Pleasantly sweet, like candy | 9.5 |
| Bromelain | 8-9 | Pleasant pineapple flavor | 9 |
| Trypsin | 3-4 | Fruity taste | 8.5 |

### Chocolate Food Bar

A chocolate flavored food bar was molded into a square shape of 2 cm length by 2 cm width by 4 mm thick. 8 mg of collagenase powder was added to the center of the food bar, and the food bar was folded in half with all borders pinched to provide a dry, dark environment for the collagenase. The 1 cm by 2 cm device made of a food bar surrounding collagenase was refrigerated until 20 min prior to use.

Following initiation of oral sex, the device was placed behind the front teeth of a human subject and secured by running the tongue against the device at the initiation of oral sex. Upon ejaculation, the food bar matrix was at least partially dissolved by running the tongue over the device, releasing the collagenase and eliminating the viscosity of the semen. The taste of the device was described as grainy, chocolate, and dense. The device was reported to last in the oral cavity for more than 10-20 minutes.

### Chocolate Flavored Caramel

A chocolate flavored caramel confection was molded into a square shape 2 cm length by 2 cm width by 3 mm thick. 8 mg of collagenase powder was added to the center of the confection, and the confection was folded in half with all borders pinched to provide a dry, dark environment for the collagenase. The 1×2 cm device made of a food bar surrounding collagenase was refrigerated until 20 min prior to use.

Following initiation of oral sex, the device was placed behind the front teeth of a human subject and secured by running the tongue against the device at the initiation of oral sex. Upon ejaculation, the confection matrix was at least partially dissolved by running the tongue over the device, releasing the collagenase and eliminating the viscosity of the semen. The taste of the device was described as “orgasmic chocolate” and dense.

### Other Embodiments

All of the features disclosed in this specification may be combined in any combination. Each feature disclosed in this specification can be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present invention, and without departing from the spirit and scope thereof, can make various changes and modifications of the invention to adapt it to various usages and conditions. Thus, other embodiments are also within the claims.

1. An edible composition for reducing semen viscosity, the composition comprising:
(i) one or more enzymes selected from the group consisting of protease, glycosidase, and a combination thereof; and
(ii) a matrix that comprises a sweetening agent;
wherein the one or more enzymes are embedded in or encapsulated by the matrix, and
wherein the matrix is dissolvable inside an oral cavity, thereby releasing the one or more enzymes.

2. (canceled)

3. The edible composition of claim 1, wherein the sweetening agent is embedded in or encapsulated by the matrix, and dissolution of the matrix releases the sweetening agent.

4. The edible composition of claim 1, wherein the matrix is a film, which encapsulates the one or more enzymes, the sweetening agent, or both.

5. (canceled)

6. The edible composition of claim 1, wherein the one or more enzymes comprises (i) one or more proteases selected from the group consisting of collagenase, bromelain, trypsin, and chymotrypsin, (ii) one or more glycosidases selected from the group consisting of amylase, sucrase, maltase, lactase, fucosidase, galactosidase, N-acetylglucosidase, N-acetylgalactosidase, N-acteylneuraminidase, N-glycolylneuraminidase, sialidase, and mannosidase, or (iii) a combination of (i) and (ii).

7. (canceled)

8. The edible composition of claim 1, wherein the sweetening agent is sucrose, fructose, glucose, galactose, maltose, sorbitol, xyitol, lactitol, maltitol, erythritol, isomalt, a sugar substitute, or a combination thereof.

9. (canceled)

10. The edible composition of claim 1, wherein the matrix comprises at least one polymer, which is dissolvable in an oral cavity, and wherein the at least one polymer is gelatin, pectin, cellulose, gum, starch, or a combination thereof.

11. (canceled)

12. The edible composition of claim 1, wherein the matrix is a food product.

13. The edible composition of claim 12, wherein the food product is a candy, gummie, gel, fruit strip, or food bar.

14. The edible composition of claim 1, further comprising one or more flavoring agents.

15. The edible composition of claim 14, wherein the flavoring agent is embedded in or encapsulated by the matrix.

16-18. (canceled)

19. The edible composition of claim 1, wherein the amount of the one or more enzymes in the edible composition ranges from about 0.1 mg to about 300 mg.

20-21. (canceled)

22. The edible composition of claim 1, wherein the amount of the sweetening agent in the edible composition ranges from about 50 mg to about 2 g.

23. (canceled)

24. A method of reducing seminal viscosity, comprising contacting an edible composition of claim 1 with a semen sample under conditions allowing for dissolution of the matrix to release the one or more enzymes.

25. The method of claim 24, wherein the contacting step is performed by placing the edible composition in an oral cavity.

26. The method of claim 25, wherein the oral cavity is a human oral cavity.

27. (canceled)

28. A method for preparing an edible composition of claim, the method comprising:
(i) providing one or more enzymes selected from the group consisting of protease, glycosidase, and a combination thereof; and
(ii) encapsulating or embedding the one or more enzymes with a film, which is dissolvable in an oral cavity.

29. The method of claim 28, wherein prior to the encapsulating or embedding step, the method further comprises mixing a sweetening agent with the one or more enzymes.

30. The method of claim 28, wherein the film comprises a sweetening agent.

31-43. (canceled)

44. A composition for reducing seminal fluid viscosity in an oral cavity, comprising:
(a) an edible semen viscosity-reducing agent, and
(b) an edible matrix that comprises a sweetening agent;
wherein the semen viscosity-reducing agent is releasably embedded in or encapsulated by the matrix, and wherein the matrix is dissolvable inside an oral cavity, thereby releasing the semen viscosity-reducing agent.

45. A method of reducing seminal fluid viscosity in an oral cavity, comprising the steps of:
(a) providing an edible composition, wherein the edible composition includes:
i. a semen viscosity-reducing agent, and
ii. a matrix that comprises a sweetening agent;
wherein the semen viscosity-reducing agent is releasably embedded in or encapsulated by the matrix, and
(b) disposing the edible composition whereby the edible composition interacts with the oral cavity, and in response to the interaction,
i. the semen viscosity-reducing agent is released from the matrix and
ii. upon contact with ambient semen in the oral cavity, the semen viscosity-reducing agent reduces the viscosity of the ambient semen.
